(19) United States
(12) Patent Application Publication
(10) Pub. No.: US 2018/0035576 A1
(43) Pub. Date: Feb. 1, 2018

(54) RADIO FREQUENCY SHIELDED TEXTILES

(71) Applicants: Ali Bostani, Montreal (CA); Farid Motamedian, Montreal (CA); Anup Kuriya, St. Lazare (CA); Raymond Waterbury, Mirabel (CA)

(72) Inventors: Ali Bostani, Montreal (CA); Farid Motamedian, Montreal (CA); Anup Kuriya, St. Lazare (CA); Raymond Waterbury, Mirabel (CA)

(21) Appl. No.: 15/346,681

(22) Filed: Nov. 8, 2016

(51) Int. Cl. H05K 9/00; G21F 3/02; H01Q 17/00

(52) U.S. Cl. CPC H05K 9/0086; H05K 9/0084; H01Q 17/005; G21F 3/02; A61B 6/107

(57) ABSTRACT

The invention is a method to modify textiles with a printing and lamination method to produce textile materials with integrated RF shielding. The shielding modifications are comprised of a network of interconnected surface patterns of periodic unit geometries organized in an arrangement to reflect RF waveforms over a range of frequencies below the 10 GHz; to shield the majority or incident RF radiation. Use of this invention enables permanent transfer of many RF shielding patterns on most textile materials for production of RF shielded consumer and industrial clothing as well as object and personal coverings.

### BACKGROUND OF THE INVENTION

Wireless RF communication of data is commonplace in the modern world; spanning tremendous usage of cell phones, countless Wi-Fi hotspots, satellite signals, radar and point-to-point underground communications. There are also the well-known heating applications of high frequency RF waves. Microwave oven exists in every home and there are numerous industrial-scale microwave applications for massive, parallel expedited heating processes.

Consequently, the impact of RF radiation sources on human health is a concern in daily life—an issue of debate since first wireless communication devices were introduced over 50 years ago. Thousands of research articles have been published about the possible health risks associated with RF radiation (1). Generally, reduced RF radiation exposure is beneficial for everyone.

The majority of the prior art discloses clothing and coverings that are augmented with lining or embedded RF shielding materials, non-specific RF wave-defeating structure, that are intended to reduce exposure to RF energy or to isolate RF energy directly. These shielding materials that are added to the garment construction are typically woven metal meshes or foils. Consequently, the resulting clothing or coverings are often bulky, awkward and costly. U.S. Pat. No. 8,434,169 B2 utilizes a metallic mesh lining layer to construct shielded clothing; U.S. Pat. No. 5,115,140 A uses a fully copper-coated textile layer to serve as a lining body shield; US 20140111363 A1 utilizes a metallic spray method to create garment pockets intended to isolate RF from carried electronic devices; U.S. Pat. No. 5,073,984 A make use of woven or knitted surface metalized fibers to create shielded clothing; Likewise, EP 1096604 B1 utilizes silver-coated fibers or yarns to produce shielded clothing.

Illustrations of more specific approaches for textile rendered, RF attenuating structures can be cited within the prior art in US2003/0224681A1 and CN 104264502 A, which disclose a textile incorporating conductive, two-dimensional periodic structures, organized as a frequency selective surface (FSS). This method establishes pass-bands and stop-bands for the only tangential elements of RF radiation. Moreover, these structures permit passage of incident waves that are oblique to the direction of periodicity, thereby limiting the RF shielding capacity of this method.

In order to produce RF shielded clothing and coverings by traditional or typical textile fabrication methods, the need exists for a simplified and versatile means to render common textile stock materials or textile products with integrated, highly effective RF shielding capacity. The approach disclosed here results in production of RF shielded clothing and object or personal covering products that are transparent to traditional or typical garment construction methods. Additionally, the resultant, apparently commonplace or normal, clothing or covering products have integrated RF shielding capacity that is transparent to the end user.

### BRIEF DESCRIPTION OF THE FIGURES

Having described the invention in general terms, reference will now be made to the accompanying drawings, which are not necessarily drawn to scale and wherein:

FIGS. 1A, 1B, 1C and 1D illustrate the various embodiments of the RF shielding patterns.

FIG. 2 illustrates a time domain data record showing the impact of the shielding material on the RF signal.

FIG. 3 illustrates RF simulation data for RF attenuation due to the shielding material across a frequency range of 1 GHz to 10 GHz.

FIG. 4 is a flow chart that illustrates the sequence of steps to apply the RF shielding patterns to textile stock material.

FIG. 5 is a flow chart that illustrates the sequence of steps to apply the RF shielding patterns to fabricated garment.

FIGS. 6A and 6B illustrate the various embodiments of the RF shielding patterns on a completed garment.

### SUMMARY OF THE INVENTION

The present invention advances the art of production of RF shielded textiles for clothing and coverings. The method of this invention centers on the innovative concept of providing a means to produce textile materials or textile products with integrated RF shielding. The invention allows for a printing and lamination process imposed onto textile materials to introduce a modification of integrated, connected surface patterns of periodic unit geometries or structures. These connected patterns serve as a high-pass RF filter, which reflects RF waveforms over a broad range of frequencies and shields the majority of incident RF radiation.

Two aspects of the invention design are integrated to establish this shielding capacity: (1) the unit geometry and spatial relationship of the pattern geometry are created in such a fashion as to establish the desired effect of disrupting and/or reflecting RF waveforms; thereby limiting propagation of tangential elements of RF radiation beyond the material, and (2) the patterns are interconnected to establish a fine Faraday cage, which is implemented with resulting opening smaller than one tenth of a quarter wavelength of the threshold RF frequency to ensure suppression of the normal incident waves.

When utilized or operated in the manner prescribed by the method stipulated herein, the use of this invention enables the transfer of many different types of RF shielding patterns on most textile materials with minimal impact on fabrication and use. Furthermore, the method of this invention is particularly suited for RF shielded consumer and industrial clothing as well as object and personal coverings.

### DETAILED DESCRIPTION OF THE INVENTION

Various embodiments of the invention are described more fully hereinafter with reference to the accompanying drawings, in which some, but not all embodiments of the invention are shown in the figures.

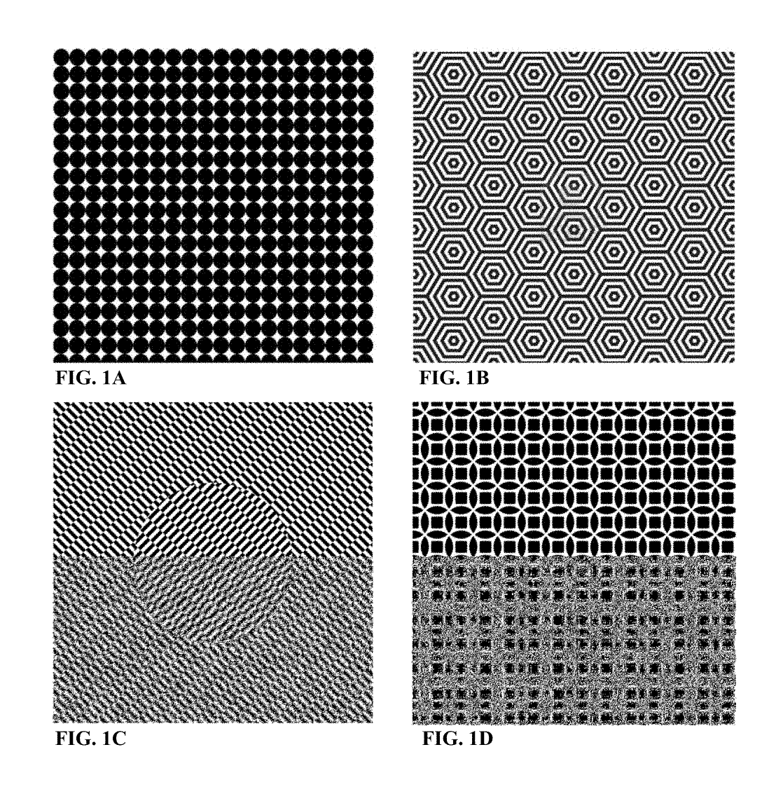

FIGS. 1A, 1B, 1C and 1D illustrate four (4) possible patterns for RF shielding periodic structures of the invention. The number of possible patterns and combinations is limitless and the patterns in these figures exemplify typical unit geometries as well as periodicity of the structures. The pattern is designed to create a Faraday cage with an embedded FSS that reflects the undesired frequency bands (2-6). The FSS is a periodic structure that is designed in a manner such that its stop-band envelopes the operating frequencies of the most common wireless devices up to the third harmonic of the band allocated for industrial, scientific and medical applications. The Faraday cage implementation is also conducted the way that up the third harmony of the industrial, scientific and medical (ISM) band is attenuated as the size of the shells of the screen are less than one tenth of the wavelengths of the waves radiated. The combination of the two attenuation techniques applied on the fabric ensures that the radiation is substantially suppressed before it reaches the object or body.

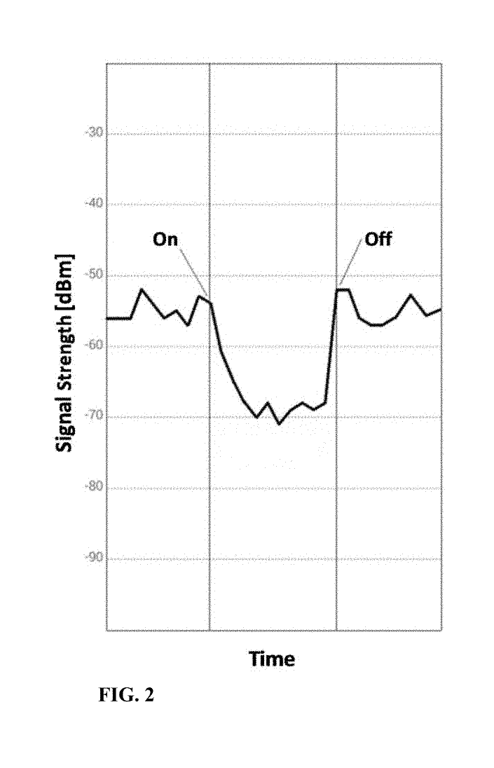

FIG. 2 is a time domain data record illustrating the typical impact of the shielding material on the RF signal; an interconnected pattern rendered on the material attenuates incident RF radiation from all directions abruptly by reflecting the undesired frequency bands. The test was completed in the presence of an RF source, radiating at a frequency of 2.45 GHz. The data record in FIG. 2 shows three phases for the signal captured by the receiving antenna: (i) the initial signal, then (ii) when it was is covered with the proposed shielded fabric for a certain period of time and, finally, (iii) the return to the initial signal level when the shielded fabric was removed. This figure clearly shows the RF attenuation, as the signal strength received by the antenna is approximately −55 dB while it is not covered, then attenuated up to −71 dB when the shielding fabric is applied.

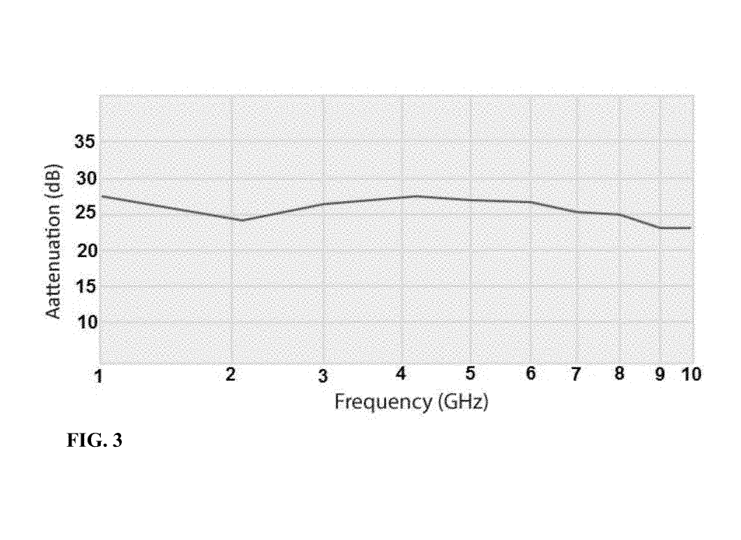

FIG. 3 shows a composite of finite element (FE) simulations of the proposed specific patterned, RF shielded fabrics across a range of frequencies to illustrate shielding effectiveness. To acquire these data, a simulation model these shielded fabrics was created having the on them, an ultra thin layer was created in FE high frequency simulation software (HFWORKS). The model was comprised of a plane to simulate the metal shielding pattern. The shielding plane was considered to be a perfect electric conductor boundary condition. The simulation was arranged as a portion of waveguide having two opposing ports with the shielding plane resting in the middle cross section of the waveguide. In this simulation arrangement, any radiation emitted from the first port must pass through the FSS plane. Therefore, radiation received at the second port is the portion that passed through the layer. Conversely, any radiation reflected back to the first port is the amount of the radiation that was blocked by the shielding plane. Therefore, measuring the S₂₁ (the scattering parameter initiated at port 1 and is received at port 2) indicates the level of the radiation attenuation achieved with the shielding pattern in place.

All simulation results were verified by iterating thought mesh refinement multiple times to ensure that both the excitation modes coming from the 2D Eigen solution at the ports as well as the scattering parameter coming from the 3D factorization had the least possible error. Furthermore, all the simulation results for shielding effectiveness were confirmed later when the fabricated prototypes were tested using a vector network analyzer.

In the most basic manual approach to the fabrication process, after potential shielding patterns are developed, the first step is printing of the shielding pattern with adhesive to the textile material.

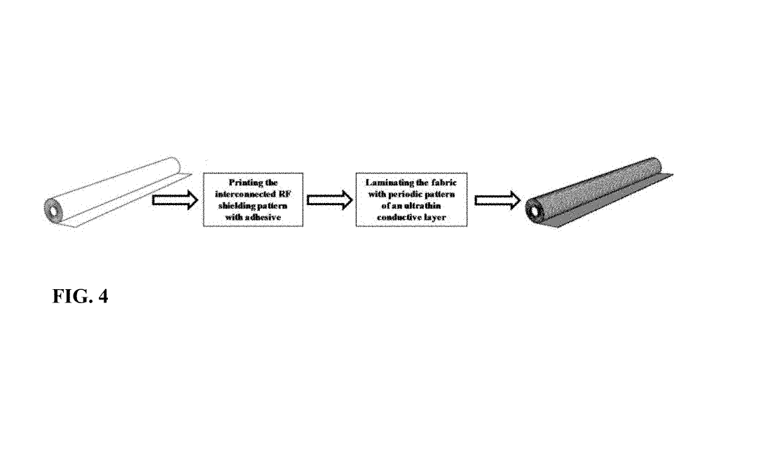

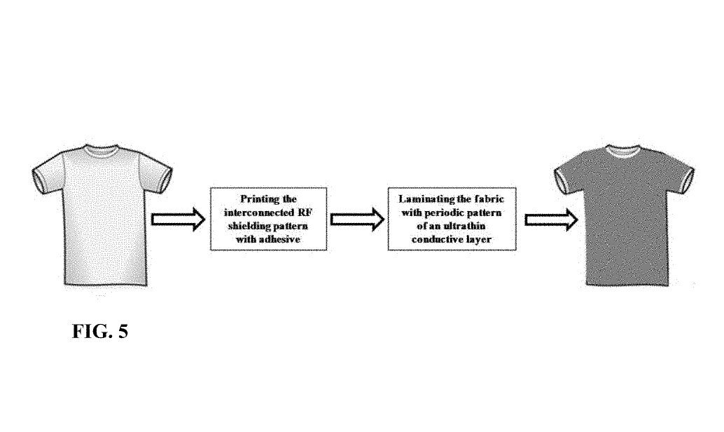

The following and final step of the process is lamination of the fabric with an ultrathin (0.1 to 100 microns) conductive sheet. After the laminating material is pressed onto the material with the printed pattern (with pressure and heat), the lamination layer captures the pattern of the printed adhesive. Afterward, the unbound lamination material is removed and the result is a textile-integrated shielding structure which will shield RF radiation, but remain thin enough so as not to compromise the flexibility and breathability of the basic fabric. FIGS. 4 and 5 illustrate, respectively, how this process implemented generally on either textile stock material or a previously fabricated garment.

For effective automated generation of production-level quantities of these RF shielded textiles with minimal processing steps and high product consistency, two alternative methods for simplified transfer of the adhesive and metal can be employed. The first utilizes a polymer film carrier with one surface holding adhesive, printed in the desired RF shielding pattern. The second utilizes a metalized film on one surface of a polymer film carrier with an adhesive, printed in the desired RF shielding pattern on top of the metalized film. Use of either prepared, pattern-rendered film reduces processing steps. The second method, in either an individual sheet format or a roll, permits direct transfer of the metalized, RF shielding patterns directly to the textile material.

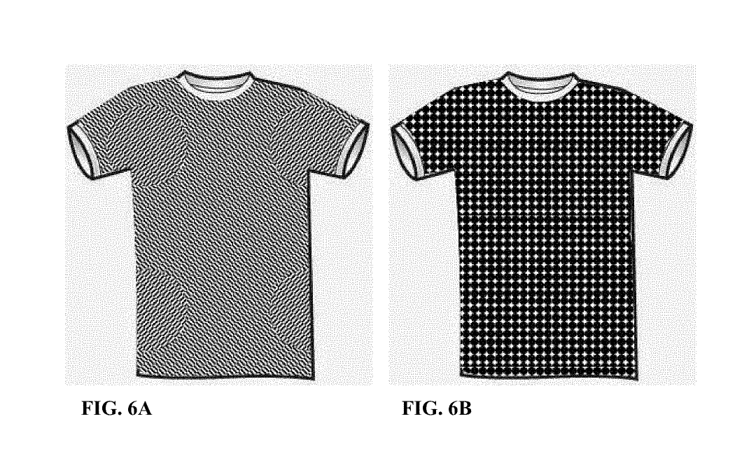

Finally, FIGS. 6A & B illustrates the various embodiments of the RF shielding patterns on a simple, completely fabricated garment in order to exemplify the product as well as the unlimited combination of shielding geometries that can be applied to garments and coverings.

### REFERENCES

1. BioInitiative Working Group, Cindy Sage and David O. Carpenter, (eds.) (2012) BioInitiative Report: A Rationale for Biologically-based Public Exposure Standards for Electromagnetic Radiation. (http://www.bioinitiative.org).
2. Bostani, A.; Webb, J. P., “A sparse finite element method for modeling evanescent modes in the stopband of periodic structures,” 14th Biennial IEEE Conference on Electromagnetic Field Computation (CEFC), pp. 1, May 2010.
3. Bostani, A.; Webb, J. P., “A Sparse Finite-Element Method for Modeling Evanescent Modes in the Stopband of Periodic Structures,” IEEE Transactions on Magnetics, vol. 47, no. 5, pp. 1186-1189, May 2011.
4. Bostani, A.; Webb, J. P., “A model-order reduction method for the passband and stopband characteristics of periodic structures,” 41st European Microwave Conference (EuMC), pp. 167-170, October 2011.
5. Bostani, A.; Webb, J. P., “Finite-Element Eigenvalue Analysis of Propagating and Evanescent Modes in 3-D Periodic Structures Using Model-Order Reduction,” IEEE Transactions on Microwave Theory and Techniques, vol. 60, no. 9, pp. 2677-2683, September 2012.
6. Wu, T.-K. (2005). Frequency Selective Surfaces. Encyclopedia of RF and Microwave Engineering. Wiley Online Library.

### FIELD OF THE INVENTION

This invention relates to a method for production of textiles having radio frequency (RF) shielding capacity.

Particularly, this invention relates to the printing and lamination methods for production of textile materials or textile products having RF shielding capacity.

More particularly, the invention relates to a method for permanent application and integration of a pattern of metal-containing, continuously connected periodic structures to textile materials. This arrangement of periodic structures reflect RF radiation wave structures over range of frequencies; rendering the resultant material for fabrication of garments or coverings fully as an RF shield.

Specifically, the invention relates to a novel technique for addition of numerous possible interconnected patterns or combination patterns of RF shielding periodic structures, or frequency selective surfaces, to textiles or textile products applicable to simple manufacturing of fully effective RF shielding garments or coverings.

Having described the invention, we claim:

1. A protective textile against radio frequency (RF) radiation comprising;
A lower layer of basic, non-conductive textile material;
An upper layer of conductive, RF radiation defeating material distributed fully in an interconnected pattern, affixed to, and supported by the basic material throughout; and
The RF radiation defeating material of the upper layer shields or protects objects covered with the textile from RF radiation coming from all directions exterior to the covered object.

2. A shielding or protective textile as claimed in claim 1 wherein the basic material includes at least one of one of natural fibers and synthetic fibers, and composites thereof.

3. A shielding or protective textile as claimed in claim 1 wherein the distributed conductive, RF radiation defeating material is comprised of metal as well as alloys and composites thereof.

4. A shielding or protective textile as claimed in claim 1 wherein the upper protective portion is affixed to the lower layer of basic textile material using one of the following methods: (1) a sequence of adhesive printing or application followed by heat transfer of metal material from a sheet of polymer carrier material; (2) direct thermal transfer of an adhesive-printed metal foil of the RF shielding pattern.

5. A shielding or protective textile, wherein the upper protective portion, comprised of an RF radiation defeating material distributed fully in an interconnected pattern, establishes a high pass filter that attenuates transmission of RE radiation below a threshold frequency of 10 GHz.

6. An RF shielding pattern as claimed in claim 5 which is a specifically engineered, integrated design of an embedded, monolayer network of a frequency selective surface of interconnected, two-dimensional periodic structures, establishing a fine Faraday cage.

7. An RF shielding pattern as claimed in claim 5, wherein the periodic, two-dimensional unit structures within an interconnected frequency selective surface network have an engineered design with openings smaller that one tenth of a quarter wavelength of the 10 GHz threshold RF attenuation frequency.

8. An RF shielding pattern as claimed in claim 5, wherein the network of interconnected frequency selective surface, two-dimensional periodic structures serves a dual, integrated purpose for suppression of the normal and tangential incident RF radiation waves.

9. An RF shielding pattern as claimed in claim 5, wherein the network of interconnected frequency selective surface, two-dimensional periodic structures is specifically engineered to suppress normal, oblique and tangential elements of incident RF radiation to ensure that all the frequencies below the 10 GHz threshold RF attenuation frequency fall into the stop band of the structure.

10. An RF shielding pattern as claimed in claim 5, wherein the network of interconnected frequency selective surface, two-dimensional periodic structures is specifically engineered to stop or reflect RF radiation, and the intensity thereof, independently of the basic material.

11. An RF shielding pattern as claimed in claim 5, wherein the network of interconnected frequency selective surface, two-dimensional periodic structures can be applied using non-uniform thicknesses of metal across the basic textile material.

12. An RF shielding pattern as claimed in claim 5, wherein the network of interconnected frequency selective surface, two-dimensional periodic structures can be applied using adhesive or solvent containing distributed, conductive, RF radiation defeating material that is comprised of metal as well as alloys and composites thereof.

13. An RF shielding pattern as claimed in claim 5, wherein the network of interconnected frequency selective surface, two-dimensional periodic structures can be repeatedly applied in a multi-layered arrangement to the basic material to create a three-dimensional RF radiation shield.

14. An RF shielding pattern as claimed in claim 5, wherein the design unit structures can be comprised of interconnected lines, dots, circles, or polygons.

15. An RF shielding pattern as claimed in claim 5, wherein the design unit structures can be comprised of or contain arbitrary figures or shapes that are solid or with openings.

16. An RF shielding pattern unit structure as claimed in claim 5, wherein the design unit structures containing arbitrary figures or shapes may be interconnected lines, dots, circles, or polygons.

17. An RF shielding pattern unit structure as claimed in claim 5, wherein the design of internal embedded features of unit structures may have a design which is engineered to complete or augment the RF shielding capacity of unit structures and/or the network of interconnected frequency selective surface, two-dimensional periodic structures.

18. An RF shielding pattern as claimed in claim 5, wherein arrangements of multiple networks of interconnected frequency selective surface, two-dimensional periodic structures can be organized uniformly or non-uniformly across the basic material.

19. An RF shielding pattern as claimed in claim 5, wherein multi-layered arrangement of the network of interconnected frequency selective surface, two-dimensional periodic structures can be organized uniformly or non-uniformly across the basic material to connect or bolster the fabric threads or weave; or interconnect subsections of similar or differing basic materials.

20. An RF shielding pattern unit structure as claimed in claim 5, wherein the design unit structures can contain internal embedded features that are not connected to the outer portion of the interconnected network of unit structures.
